(19) United States
(12) Patent Application Publication
(10) Pub. No.: US 2024/0207518 A1
(43) Pub. Date: Jun. 27, 2024

(54) Housing Components for an Injection Device

(71) Applicant: Sanofi, PARIS (FR)

(72) Inventors: Beate Franke, Frankfurt am Main (DE); Christian Frescha, Rüsselsheim (DE); Michael Helmer, Frankfurt am Main (DE); Stephan Muecke, Rüsselsheim (DE); Peter Nober, Rüsselsheim (DE); Matthias Rau, Rüsselsheim (DE); Michael Schabbach, Frankfurt am Main (DE); Martin Vitt, Frankfurt am Main (DE)

(21) Appl. No.: 18/288,659

(22) PCT Filed: May 2, 2022
(86) PCT No.: PCT/EP2022/061647

§ 371 Date: Oct. 27, 2023

(30) Foreign Application Priority Data
May 3, 2021 (EP) 21315077.4

(51) Int. Cl. A61M 5/20

(52) U.S. Cl. CPC A61M 5/20

(57) ABSTRACT

A housing for a drug delivery device includes a first housing component configured to accommodate a cartridge filled with a medicament and including a first connecting end; a second housing component configured to accommodate a drive mechanism of the drug delivery device and including a second connecting end; an insert provided on one of the first connecting end and the second connecting end; a receptacle confined by a sidewall and provided on the other one of the first connecting end and the second connecting end; a fastening element provided on the insert; a counter fastening element complementarily shaped to the fastening element and rotationally supported on the sidewall between a locking position and a release position; a mechanical coding provided on the insert and comprising a coding feature; and a mechanical counter coding provided in the receptacle and comprising a counter coding feature.

### CROSS REFERENCE TO RELATED APPLICATIONS

The present application is the national stage entry of International Patent Application No. PCT/EP2022/061647, filed on May 2, 2022, and claims priority to Application No. EP 21315077.4, filed on May 3, 2021, the disclosures of which are incorporated herein by reference.

### TECHNICAL FIELD

The present disclosure relates to the field of drug delivery devices and systems, particularly to injection devices for injecting a liquid medicament. More particularly, the present disclosure is generally directed to drug delivery devices and systems comprising a multi-component housing, wherein one housing component is configured to accommodate a medicament container, such as a cartridge and wherein another housing component is configured to accommodate a drive mechanism to operably engage with the medicament container for expelling or withdrawing a dose of the medicament.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump. With respect to reusable injection or delivery devices, a patient may have to load or to replace a cartridge.

Reusable injection devices typically comprise a multi-component housing. For instance, the housing may comprise a proximal housing component, such as a body and a distal housing component, such as a cartridge holder detachably connectable to the body. Once a medicament provided in a medicament container, such as a cartridge, is empty, the cartridge holder may be disconnected from the body of the injection device and the empty cartridge may be removed and replaced with a new cartridge.

Another concern may arise from cartridges being manufactured in essentially standard sizes and manufactured to comply with certain recognized local and international standards. Consequently, such cartridges are typically supplied in standard sized cartridges (e.g. 3 ml cartridges). Therefore, there may be a variety of cartridges supplied by a number of different suppliers and containing a different medicament but fitting a single drug delivery device. As just one example, a first cartridge containing a first medicament from a first supplier may fit a drug delivery device provided by a second supplier. As such, a user might be able to load an incorrect medicament into a drug delivery device and, then, dispense said medicament (such as a rapid or basal type of insulin) without being aware that the medical delivery device was perhaps neither designed nor intended to be used with such a cartridge.

As such, there is a growing desire from users, health care providers, caregivers, regulatory entities, and medical device suppliers to reduce the potential risk of a user loading an incorrect drug type into a drug delivery device. It is also desirable to reduce the risk of dispensing an incorrect medicament (or the wrong concentration of the medicament) from such a drug delivery device.

There is, therefore, a general need to physically dedicate or mechanically code a cartridge and/or cartridge holder to its drug type and design an injection device that only accepts or works with the dedication or coded features provided on or with the cartridge and/or cartridge holder so as to prevent unwanted cartridge cross use. Similarly, there is also a general need for a dedicated cartridge that allows the medical delivery device to be used with only an authorized cartridge containing a specific medicament while also preventing undesired cartridge cross use.

With drug delivery devices comprising a multi-component housing, e.g. having a first and a second housing component, it is also desirable to provide a failure safe and well-defined mechanical connection between the housing components, which may be detachably or non-detachably connectable. Here, the present disclosure aims to provide an improvement for connecting and disconnecting first and second housing components of a drug delivery device.

### SUMMARY

In one aspect the disclosure relates to a housing of a drug delivery device, in particular to a housing of an injection device, such as a handheld injection pen. The housing comprises a first housing component configured to accommodate a cartridge filled with a medicament. The first housing component comprises a first connecting end. The housing further comprises a second housing component. The second housing component is configured to accommodate a drive mechanism of the drug delivery device. Typically, the drive mechanism comprises a piston rod extending in longitudinal direction and configured to operably engage with a piston or bung of the cartridge for expelling a dose of the medicament from the cartridge.

The second housing component comprises a second connecting end. Typically, the first connecting end is connectable to the second connecting end to form or to constitute the housing of the drug delivery device. With some examples the first housing component is an elongated or tubular shaped housing component comprising the first connecting end at a longitudinal proximal end. The second housing component may be also of tubular or elongated shape. The second connecting end may be located at a distal longitudinal end of the second housing component.

There is further provided an insert on one of the first connecting end and the second connecting end. The insert is typically integrally formed with the respective first or second housing component. There is further provided a receptacle on the other one of the first connecting end and the second connecting end. The receptacle is confined by a sidewall of the other one of the first connecting end and the second connecting end. The insert is insertable into the receptacle along the longitudinal direction for mutually fastening the first housing component and the second housing component and/or for forming or establishing the housing of the drug delivery device. Typically, the receptacle is provided at one of the first and second connecting ends and forms a respective connecting end. The insert is provided on the other one of the first and second connecting ends and forms a respective connecting end.

The receptacle comprises an inner cross-section sized and shaped to receive the insert therein. Typically, an inside diameter or inside cross-section of the receptacle closely matches an outside diameter or outer cross-section of the insert.

The housing comprises a fastening element provided on the insert. The housing further comprises a counter fastening element complementary shaped to the fastening element and rotationally supported on the sidewall between a blocking position and a release position. The fastening element and the counter fastening element are mutually engageable in order to fix the insert to the receptacle. Hence, by way of the fastening element mechanically engaged with the complementary-shaped counter fastening element the first housing component can be fixed to the second housing component; and vice versa.

Typically, mutual engagement of the fastening element and the counter fastening element is obtained by rotating or pivoting the counter fastening element from the release position into the locking position. Here, the counter fastening element may be rotated or pivoted relative to the fastening element. Typically, the fastening element and the counter fastening element enable and support a two-stage or two-step assembly of the first and second housing components. During a first step of assembly the insert is longitudinally inserted into the receptacle until a final insert position has been reached. During this first assembly step the counter fastening element is and remains in the release position. When or after reaching the insert position the counter fastening element is rotatable or pivotable from the release position towards and into the locking position, thereby bringing the fastening element and the counter fastening element in mutual engagement for fixing the first and second housing components relative to each other.

According to a further example the counter fastening element is rotatable or movable into the locking position only when the insert has reached the final insert position inside the receptacle. In this way, there is provided a rather direct and unequivocal feedback to a user when the mechanical coding does not match the mechanical counter coding. Then, engaging the fastening element with a counter fastening element and/or rotating or moving of the counter fastening element into the locking position may be permanently blocked.

Hence, mutual interaction of the fastening element and the counter fastening element may provide a functionality that the counter fastening element is rotatable or movable into the locking position only when the insert has reached the final insert position inside the receptacle. In this way it is ensured, that a mechanical connection between the first housing component and the second housing component can only be established when the insert is fully and/or correctly inserted in to the receptacle.

According to a further example and with all other longitudinal positions of the insert inside the receptacle before reaching the final insert position a movement of the counter fastening element from the release position towards the locking position may be blocked. In this way, a premature and unintended transfer of the counter fastening element from the release position towards and/or into the locking position can be effectively prevented.

The housing further comprises a mechanical coding provided on the insert. The mechanical coding comprising at least one coding feature. The housing further comprises a mechanical counter coding provided in the receptacle and comprising at least one counter coding feature.

The mechanical coding is complementary or correspondingly shaped to the mechanical counter coding and together with the mechanical counter coding it may form or constitute a matching pair of a coding and a counter coding of a common type. When the mechanical coding does not match the mechanical counter coding, i.e. when the mechanical coding is of a first type and when the mechanical counter coding is of a second type the mechanical coding and the mechanical counter coding are configured and hence operable to prevent an engagement of the fastening element with the counter fastening element and/or to prevent an insertion of the insert into the receptacle. In either way and with a non-matching pair of a mechanical coding and a mechanical counter coding a mutual assembly and/or fixing of the first and second housing components is effectively prevented.

Generally, a mutual assembly of the first housing component and the second housing component is only possible when the first housing component is provided with a mechanical coding that matches a complementary-shaped mechanical counter coding of the second housing component.

In this way different housings for different drug delivery devices can be provided having first and second housing components that are of similar or even identical outer shape but distinguish by way of a coding and a counter coding. In this way, unintended cross use of e.g. a first housing component of a housing of a first type with a second housing component of a housing of a second type can be effectively prevented. It is only when the mechanical coding of the insert matches the mechanical counter coding in the receptacle that the mechanical connection between first and second housing components can be established to mutually connect and to mutually fix the first and second housing components. With all other pairings or combinations of a mechanical coding, e.g. a mechanical coding of a first type with a non-matching mechanical counter coding, e.g. of a second or third type, the insert cannot be fixed to or inserted into the receptacle. Then, the mutual assembly and/or fixing of the first and the second housing components is effectively prevented.

Prevention of the mutual engagement of the first connecting end with the second connecting end of non-matching housing components can be effectively achieved in at least two different ways. According to some examples the mechanical coding and the non-matching mechanical counter coding are configured to prevent at least a partial insertion or a complete insertion of the insert into the receptacle along the longitudinal direction. Here, and with a coding on the insert non-matching with the counter coding of the receptacle the insert may be mechanically blocked from entering the receptacle. Alternatively, the insert may be sized and shaped to enter the receptacle but then the coding non-matching with the counter coding is configured to prevent an engagement of the fastening element with the counter fastening element. In effect, for a coding non-matching with a counter coding the first housing component cannot be connected or fixed to the second housing component.

According to a further example of the housing one of the fastening element and the counter fastening element comprises a radial protrusion. The other one of the fastening element and the counter fastening element comprises a fastening groove. With some examples the insert comprises a radial protrusion on an outside surface and the counter fastening element comprises a complementary-shaped fastening groove on an inside facing surface. With other examples the fastening element comprises the fastening groove on an outside surface of a sidewall of the insert and the counter fastening element comprises a complementary shaped radial protrusion on an inside surface. Here, the radial protrusion protrudes and extends radially inwardly from an inside surface of the counter fastening element.

Providing of a radial protrusion and providing of a fastening groove on mutually corresponding side wall sections of the insert and the housing, respectively, provides and allows a nested arrangement of the insert inside the receptacle. Typically, and when providing the fastening element on the outside surface of the insert and when providing at least a portion of the counter fastening element mechanically engaging with the fastening element on the inside of the receptacle the mutually interacting mechanical parts of the counter fastening element and the fastening element are not visible from outside the housing. In this way the mutually engaging mechanical components of the fastening element and the counter fastening element are concealed and do not contribute to the outer appearance of the housing.

Typically, the fastening groove comprises a radial depth that is smaller than a radial thickness of a side wall of the respective component, in which the fastening groove is provided. When the fastening groove is provided on a sidewall of the insert, the radial depth of the fastening groove is smaller than the thickness of the sidewall of the insert. In this way, the fastening groove may only have a minimal influence on a structural weakening of the respective housing component. The fastening groove is void of a through opening extending radially through a sidewall of the respective housing component. In this way, the mechanical rigidity and stability of the respective housing component provided with the fastening groove can be improved compared to solutions, wherein a groove or the like fastening structure extends entirely through the sidewall of a respective housing component.

According to a further example the fastening groove comprises a first groove portion and a second groove portion. The first groove portion extends along the longitudinal direction and the second groove portion extends along a circumferential direction. The second groove portion merges into the first groove portion.

By providing a groove with a first groove portion and a second groove portion and kind of a L-shaped groove can be provided for one of the fastening element and the counter fastening element. The groove provides and defines a two-step assembly process. Typically, the radial protrusion of one of the fastening element and the counter fastening element is shaped and/or configured to slide along the first groove portion during a first step or first phase of assembly. During this first step of assembly the counter fastening element is and remains in the release position, thus providing an unhindered longitudinal sliding of the radial protrusion along the first groove portion.

When the radial protrusion reaches an end position inside the first groove portion the insert and the receptacle are typically in a final insert position. Then and during a second step of assembly the counter fastening element is rotatable from the release position towards and into the locking position, e.g. by rotating the counter fastening element in circumferential or tangential direction relative to the sidewall. During this second step of assembly the radial protrusion slides along the second groove portion. In effect and during the first step of assembly the radial protrusion is subject to a sliding movement along a first direction. During the second step of assembly the radial protrusion is subject to a sliding movement along a second direction. The first and the second directions are typically defined by the shape of the fastening groove. The first direction extending along the extent of the first groove portion may be perpendicular to the second direction extending along the extent of the second groove portion.

Typically and with further examples, the first and second housing components may be rotationally locked relative to each other by the mechanical coding engaged with the mechanical counter coding. Here, the mechanical coding and the mechanical counter coding may provide a twofold or double function. In a first aspect, the mechanical coding and the mechanical counter coding may prevent assembly of a non-matching pair of first and second housing components belonging to housings of a drug delivery devices of different types. In a second aspect the mechanical coding and mechanical counter coding may comprise, form or constitute a keyed engagement of the insert and of the receptacle, thereby rotationally locking the insert to the receptacle and hence thereby rotationally locking the first housing component to the second housing component upon insertion of the insert into the receptacle in longitudinal direction.

According to a further example the first groove portion adjoins a longitudinal end face of one of the first connecting end and the second connecting end. Typically, the first groove portion adjoins a longitudinal end face of a longitudinal end of a sidewall of the second housing component or first housing component. When the fastening groove is provided on the insert of the first housing component the first groove portion adjoins a proximal end face of a sidewall of the first housing component. When the groove is provided on the second housing component the first groove portion adjoins a distally located longitudinal end face of the sidewall of the second housing component.

With the fastening groove adjoining to a longitudinal end face of one of the first connecting end and the second connecting end the complementary shaped radial protrusion of the other one of the first connecting end and the second connecting end may easily engage and enter the respective first groove portion upon insertion of the insert into the receptacle. This provides a rather smooth longitudinal sliding and insertion of the first housing component relative to the second housing component, e.g. during the first step of assembly.

According to a further example the second groove portion merges into the first groove portion at a longitudinal distance from the longitudinal end face of one of the first and second connecting ends. Typically, the second groove portion extends along a circumferential or transverse direction with regard to a tubular shape of the first and/or the second housing component. For a radial projection or protrusion configured to engage with the first and/or second groove portions it is required and intended that the projection or protrusion is circumferentially aligned with the second groove portion when the first and second housing components reach the final insert position.

With some examples the second groove portion merges into a longitudinal end of the first groove portion. The longitudinal end of the first groove portion is typically located at a longitudinal offset from one of the first and second connecting ends. With some examples the first groove portion is of rather straight and elongated shape. It has a first longitudinal end coinciding with the longitudinal end face of one of the first and second connecting ends of one of the first or second housing components, respectively. The first groove portion comprises a second longitudinal end that merges into the second groove portion. The second longitudinal end of the first groove portion is located opposite the first longitudinal end of the first groove portion.

In this way a L-shaped groove with first and second groove portions extending substantially perpendicular to each other can be provided.

According to a further example the fastening element comprises a snap element. The counter fastening element comprises a counter snap element to engage with the snap element when the counter fastening element reaches the locking position. The snap element and the counter snap element are shaped and configured to establish or contribute to a form-fitting engagement between the fastening element and the counter fastening element. Here, one of the snap element and the counter snap element can be implemented as a radial protrusion and the other one of the snap element and the counter snap element can be configured as a radial recess to receive the radial protrusion or projection.

Mutual engagement of the snap element and the counter snap element may come along with an audible, tactile or otherwise palpable feedback for the user rotating the counter fastening element towards and into the locking position. Such a palpable feedback signal provided by the mutual engagement of the snap element and the counter snap element is beneficial to indicate to the user, that the locking position of the counter fastening element has been reached and that the first and second housing components are securely attached to each other.

According to a further example at least one of the snap element and the counter snap element is elastically deformable in radial direction. This is of particular benefit when the snap element and the counter snap element comprise a radial protrusion and a radial recess being complementary shaped.

According to a further example one of the snap element and the counter snap element is located in the second groove portion. In this way, a respective mutual engagement can be obtained when the radial protrusion of one of the fastening element and the counter fastening element slides along the second groove portion until it engages the complementary shaped snap element or counter snap element.

With some examples, the snap element is located in the second groove portion. It may be provided on a bottom or on a sidewall or side edge of the second groove portion. For example, the snap element may be implemented as a radial recess or radial protrusion in the bottom of the second groove portion. Alternatively, the snap element may be implemented as a protrusion protruding inwardly from the sidewall or side edge of the second groove portion. Implementing of the snap element inside the second groove portion provides numerous possibilities and options for a rather compact design of the respective snap element.

According to a further example one of the snap element and the counter snap element is located at a longitudinal end of the second groove portion, which is located opposite the first groove portion. The longitudinal end of the second groove portion may provide or form a dead end of the second groove portion. It may define a first circumferential end of the second groove portion located opposite to a second circumferential end of the second groove portion. Typically, the second circumferential end of the second groove portion may merge with the first groove portion. Here, and since the second groove portion extends in circumferential direction, the longitudinal end of the second groove portion may be located circumferentially and/or tangentially offset from the first groove portion.

According to another example the snap element comprises a radial recess in the second groove portion and the counter snap element comprises a radial protrusion to engage with the radial recess of the snap element. Here, and with some examples the counter snap element may coincide with a radial protrusion of the counter fastening element. With an alternative example the snap element comprises a protrusion in the second groove portion and the counter snap element comprises a complementary-shaped recess to engage with the radial protrusion of the snap element. Here, the snap element may comprise a protrusion protruding inwardly either from the bottom of the second groove portion or from a side edge of the second groove portion to engage with a complementary shaped recessed structure of the counter snap element shaped and configured to slide along the second groove portion.

According to a further example the sidewall comprises a first retainer operable to fix the counter fastening element in the release position. By way of the first retainer the counter fastening element can be kept in the release position. In this way, an uncontrolled or self-acting rotation or movement of the counter fastening element relative to the sidewall can be effectively prevented. This might be of particular benefit when the first and second housing components are disconnected. When disconnected the first retainer is operable to keep the counter fastening element in the release position. For inserting the insert into the receptacle with a concurrent insertion of the radial protrusion into the first groove portion it may be required that the counter fastening element is and remains in the release position. With the first retainer it can be ensured, that the counter fastening element is and remains in the release position. Then, a smooth insertion of the insert into the receptacle accompanied by an insertion of the radial protrusion into and along the first groove portion can be provided.

According to a further example the sidewall comprises a second retainer operable to fix the counter fastening element in the locking position. In this way an uncontrolled or self-acting movement of the counter fastening element from the locking position towards the release position can be effectively prevented. Hence, an uncontrolled release of the fastening element and the counter fastening element can be effectively prevented.

The second retainer may be separated from the first retainer along the circumference of the sidewall confining the receptacle. In this way and when the sidewall is provided with a first and with a second retainer the counter fastening element can be fixed in the release position as well as in the locking position.

According to a further example the counter fastening element comprises a counter retainer complementary shaped to at least one of the first retainer and the second retainer. The counter retainer is configured to form a snap fit connection with at least one of the first retainer and the second retainer. When the sidewall is provided with a first and a second retainer the counter retainer is shaped and configured to selectively engage with both, the first retainer and the second retainer when the counter fastening element is in one of the locking position and the release position.

The snap fit connection between the counter retainer and at least one of the first and second retainers may provide an audible and/or haptic feedback to a user, that the counter fastening element is in one of the locking position and the release position. In this way, a well-defined feedback is provided to the user with regard to the momentary status, location or configuration of the counter fastening element.

According to a further example the first retainer and the second retainer may comprise a somewhat equal shape or geometry. They may equally and mechanically engage with the counter retainer.

With some examples the first retainer and the second retainer may distinguish with regard to their cross-sectional shape or geometry. When the first and the second retainer comprise different geometries there can be provided different engaging and/or release forces for establishing and/or abrogating a snap fit connection of the counter retainer with any of the first and second retainers. For instance, a force to establish a snap fit connection between the counter retainer and the first retainer may be smaller than a force required for establishing or releasing a snap fit connection between the counter retainer and the second retainer. In this way, there can be provided different release forces for transferring the counter fastening element from the locking position towards the release position. This force may be substantially higher compared to a force required for transferring the counter fastening element from the release position towards and into the locking position. Then, establishing of a mutual fastening of first and second housing components may be easier than abrogating the mutual connection.

According to a further example one of the first retainer, the second retainer and the counter retainer comprises a radial protrusion. The other one of the first retainer, the second retainer and the counter retainer comprises a radial recess complementary shaped to the radial protrusion. When the radial recess engages the radial protrusion the counter retainer is engaged with one of the first and second retainers. Typically, the first retainer and the second retainer comprise a snapfit feature of common type. So, when the counter retainer comprises a radial protrusion the first retainer and the second retainer both comprise a radial recess. When the counter retainer comprises a radial recess, both, the first retainer and the second retainer each comprise a radial protrusion.

With some examples the first retainer and the second retainer are provided on an outside surface of the sidewall confining the receptacle. They may be located on the outside of the second housing component. The counter retainer is provided on an inside surface of the counter fastening element. The counter fastening element may comprise a sleeve or a ring surrounding the sidewall confining the receptacle. Here, the counter retainer may comprise a radial protrusion protruding radially inwardly from a sidewall of the counter fastening element, e.g. protruding inwardly from a sidewall of the sleeve or ring of the counter fastening element.

According to a further example the housing comprises a third retainer located in the second groove portion and being operable to fix the counter fastening element in the locking position. By way of the third retainer the second retainer may become superfluous. Hence, the third retainer may be provided instead of the second retainer.

With further examples the third retainer may be provided by the snap element that is located in the second groove portion. The counter snap element engaging the snap element being operable to fix the counter fastening element in the locking position. With this example but also generally, it is of particular benefit, when the mechanical coding and the mechanical counter coding provide a rotational lock for the first and second housing components. Here, the mechanical coding may be in a keyed engagement with the mechanical counter coding. The keyed engagement may provide a longitudinal sliding movement of the first housing component relative to the second housing component but prevents and/or impedes a rotation of the first housing component relative to the second housing component.

According to another example the fastening element comprises the fastening groove that is provided on an outside surface of the insert. With the fastening groove provided on an outside surface of the insert the insert may be void of any radially outwardly protruding projections. This allows and supports a rather easy insertion of the insert into the receptacle in longitudinal direction. As mentioned above, the fastening groove may comprise a radial depth that is smaller than the thickness of the sidewall of the insert. In this way, the insert may be void of through openings or through recesses in its sidewall. Accordingly, the mechanical stability and rigidity or stiffness of the insert can be improved.

According to a further example the counter fastening element comprises a locking ring or locking sleeve rotationally supported on an outside surface of the sidewall that confines the receptacle. With some examples the locking ring may be provided on the outside surface of the sidewall of the second housing component. The locking ring or locking sleeve may be longitudinally fixed or constrained to the sidewall confining the receptacle, e.g. it may be axially locked to the second connecting end. It may be hindered from moving in longitudinal direction relative to the sidewall. It may rotate or pivot relative to the sidewall with an axis of rotation extending substantially parallel to a symmetry axis of the tubular shaped housing component or connecting end.

Providing the locking ring on an outside surface of the sidewall confining the receptacle is beneficial to provide a direct and immediate access to the locking ring. It may be operated, handled and hence rotated by a user from outside the housing.

According to a further example the counter fastening element comprises the radial protrusion. The radial protrusion extends radially inwardly through a circumferentially extending slot of the sidewall of the second housing component. Typically, the radial protrusion protrudes and extents radially inwardly from an inside surface of a sidewall of the locking ring or locking sleeve. The radial extent of the radial protrusion is larger than a thickness of the sidewall of the second housing component, at least in a region being in direct vicinity of the slot through which the radial protrusion extends.

In this way and when the locking ring or a locking sleeve is assembled on the sidewall the radial protrusion extends radially through the circumferentially extending slot to such an extent, that a free end of the radial protrusion protrudes radially inwardly from an inside surface of the sidewall and extents radially into the receptacle. At least the portion of the radial protrusion protruding radially inwardly from the sidewall of the receptacle is configured and shaped to engage the fastening groove provided on the outside surface of the insert.

According to a further example the sidewall of the second housing component comprises a fastening structure by way of which the counter fastening element is longitudinally constrained or longitudinally fixed to the housing. By way of the fastening structure the counter fastening element can be secured and permanently attached to the sidewall confining the receptacle.

According to a further example the fastening structure comprises a longitudinal assembly groove adjoining a longitudinal end face of the sidewall and merging into the circumferentially extending slot of the fastening structure. The assembly groove may provide a longitudinal sliding movement of the radially inwardly extending protrusion of the counter fastening element upon assembly of the counter fastening element to the sidewall. The longitudinal assembly groove may comprise a radial depth that is smaller than or equal to the thickness of the side wall. The longitudinal assembly groove merges into the circumferentially extending slot. The circumferentially extending slot comprises a through opening extending through the sidewall of the receptacle. During and/or for assembly of the counter fastening element to the fastening structure the radially inwardly extending protrusion of the counter fastening element may be subject to an elastic deformation as it slides along the longitudinally extending assembly groove.

According to a further example the fastening structure comprises a beveled snap element arranged in the assembly groove and adjoining the circumferentially extending slot. Typically, the beveled snap element narrows the radial depth of the assembly groove towards the circumferentially extending slot. Insofar and during an assembly process for fitting the counter fastening element to the sidewall the radial protrusion of the counter fastening element and/or sidewall is subject to an increasing elastic deformation. Once the radial protrusion has passed the beveled snap element, e.g. a respective ramp of the fastening structure the radial protrusion of the counter fastening element may audibly relax and clip into the circumferentially extending slot. In this way, an audible and/or haptic feedback is provided when the counter fastening element reaches a final assembly configuration at the fastening structure. By way of the beveled snap element a return or disassembly movement of the counter fastening element through the assembly groove is effectively impeded and prevented.

According to a further example one of the coding feature and the counter coding feature comprises a radial coding projection. The other one of the coding feature and the counter coding feature comprises a longitudinally extending coding slot shaped to slidably receive the radical coding projection. With a matching pair of a coding feature and a counter coding feature a transverse cross-section of the coding feature matches with or corresponds to the transverse cross-section of the counter coding feature. In other words, the cross-section of the coding projection matches the cross-section of the coding slot. The coding slot extends in longitudinal direction, hence parallel to the symmetry axis of the first or second housing component. In this way, the mutual engagement between the coding projection and the coding slot supports and enables a longitudinal sliding movement of the insert into the receptacle. By way of the mutually engaged coding slot and coding projection a rotational interlock between the insert and the receptacle can be provided.

According to another example the mechanical coding is defined by at least one of: a number of coding features, a circumferential position of coding features on the insert, a circumferential extent of coding features on the insert, a radial extent of coding features on the insert and a cross-sectional geometry or shape of coding features in a plane transverse to the longitudinal direction (z).

Complementary or correspondingly and according to further examples the mechanical counter coding is defined by at least one of: a number of counter coding features, a circumferential position of counter coding features in the receptacle, a circumferential extent of counter coding features in the receptacle, a radial extent of counter coding features in the receptacle and a cross-sectional geometry or shape of counter coding features in a plane transverse to the longitudinal direction (z).

With a matching pair of a coding and a counter coding the number of coding features equals the number of counter coding features. Furthermore, the circumferential position of the coding features on the insert matches and corresponds with the circumferential position of the counter coding features in the receptacle. The circumferential extent of coding features on the insert matches and corresponds with the circumferential extent of the counter coding features in the receptacle. Furthermore, the radial extent of the coding features on the insert matches and corresponds to the radial extent of the counter coding features in the receptacle and a cross-sectional geometry or shape of the coding features on the insert matches and corresponds to the cross-sectional geometry or shape of the counter coding features in a plane transverse to the longitudinal direction (z).

With a non-matching pair of a coding and a counter coding, at least one of the number of coding features on the insert, a circumferential extent of coding features on the insert, a radial extent of coding features on the insert and a cross-sectional geometry or shape of coding features in a plane transverse to the longitudinal direction (z) does not match with at least one of the respective number of counter coding features, a circumferential position of counter coding features in the receptacle, a circumferential extent of counter coding features in the receptacle, a radial extent of counter coding features in the receptacle or a cross-sectional geometry or shape of counter coding features in a plane transverse to the longitudinal direction (z). With a coding non-matching a counter coding insertion of the radial coding projection into the coding slot is effectively prevented.

According to another aspect there is provided an injection device for injecting a dose of a medicament. The injection device comprises a housing as described above and a cartridge arranged inside the housing. The cartridge comprises a barrel filled with a medicament and sealed in a proximal longitudinal direction by a movable bung. The injection device further comprises a drive mechanism arranged inside the housing. The drive mechanism comprises a piston rod operable to exert a distally directed dispensing force onto the bung of the cartridge. Typically, the injection device is implemented as a hand held or portable injection device. The injection device may comprise a pen-type injector.

With some examples the receptacle is provided as a housing insert fixedly attachable or fixedly attached to an elongated housing component, e.g. the first or second housing component of the housing of the drug delivery device. The housing insert may be rotationally and/or longitudinally fixed to the elongated housing component. Insofar all features and benefits as described above in connection with the receptacle equally apply to a housing insert fixedly connectable or fixedly connected to a respective housing component.

In another aspect the disclosure relates to a housing of a drug delivery device, e.g. of an injection device. Also here, the housing comprises a first housing component configured to accommodate a cartridge filled with a medicament. The first housing component comprises a first connecting end. The housing further comprises a second housing component. The second housing component is configured to accommodate a drive mechanism of the drug delivery device. Typically, the drive mechanism comprises a piston rod extending in longitudinal direction and configured to operably engage with a piston or bung of the cartridge for expelling a dose of the medicament from the cartridge.

The second housing component comprises a second connecting end. Typically, the first connecting end is connectable to the second connecting end to form or to constitute the housing of the drug delivery device. There is further provided an insert on one of the first connecting end and the second connecting end. The insert is typically integrally formed with the respective first or second housing component. There is further provided a receptacle on the other one of the first connecting end and the second connecting end. The insert is insertable into the receptacle along the longitudinal direction for mutually fastening the first housing component and the second housing component and/or for forming or establishing the housing of the drug delivery device. Typically, the receptacle is provided at one of the first and second connecting ends and forms a respective connecting end. The insert is provided on the other one of the first and second connecting ends and forms a respective connecting end.

The receptacle comprises an inner cross-section sized and shaped to receive the insert therein. Typically, an inside diameter or inside cross-section of the receptacle closely matches an outside diameter or outer cross-section of the insert.

The housing further comprise a fastening element provided on the insert and a counter fastening element complementary shaped to the fastening element and provided on a sidewall confining the receptacle. The counter fastening element is rotationally supported on the sidewall. It is rotatable or pivotable between a locking position and a release position as described above.

The housing according to the present aspect may be void of a mechanical coding and may be void of a mechanical counter coding. In general, the housing according to the present aspect comprises first and second housing components as described above with the exception of being void of a mechanical coding and a mechanical counter coding.

According to another aspect the disclosure relates to a kit of at least a first housing as described above and a second housing as described above. The coding feature of the first housing distinguishes from the coding feature of the second housing with regard to at least one a number of coding features, a circumferential position of the coding features on the insert, a circumferential extent of coding features on the insert, a radial extent of coding features on the insert, and a cross-sectional geometry or shape of coding features in a plane transverse to the longitudinal direction (z).

Correspondingly, the counter coding feature of the first housing distinguishes from the counter coding feature of the second housing with regard to at least one a number of counter coding features, a circumferential position of the counter coding features in the receptacle, a circumferential extent of the counter coding features in the receptacle, a radial extent of the counter coding features in the receptacle, and a cross-sectional geometry or shape of the counter coding features in a plane transverse to the longitudinal direction (z).

The first housing is provided with a pair of a coding and a counter coding of a first type. The second housing is provided with a pair of a coding and a counter coding of a second type. The coding of the first type is unable to pair or to engage the counter coding of the second type. Vice versa, the counter coding of the first type is unable to pair or to engage with the coding of the second type. In this way the first housing component of the first housing is unable to pair or to connect with the second housing component of the second housing. Unintended cross-use of first and second housing components provided and equipped with different and hence non-matching codings and counter codings can be effectively prevented and impeded.

It is only the coding of the first type which is able and configured to pair or to engage with the counter coding of the first type. The coding of the second type is only and exclusively engageable or connectable to the counter coding of the second type; and vice versa.

Generally, and with some examples the first housing components of different housings may distinguish by the size and/or geometry of an accommodating space for receiving a medicament container or cartridge. In particular, a housing with a coding of a first type may be exclusively equipped with a first cartridge or medicament container. A housing with a coding of a second type may be exclusively equipped with a cartridge or a second medicament container. For this, medicament containers, cartridges as well as the interior of the first housing components may comprise further codings or coding features or may distinguish with regard to their size or geometry such that only one dedicated cartridge or medicament container unequivocally fits into only one dedicated first housing component.

With some examples, the first housing component is provided with a mechanical coding to engage with a complementary shaped counter coding of a cartridge. With further examples the first housing component may be provided with at least one of an electronic, a visual or optical coding configured to match with a complementary counter coding of the cartridge, which is also of electronic, visual or optical type.

Moreover, at least one of the cartridge and the first housing component may be provided with a locking or fastening feature by way of which a cartridge can be fixed and/or retained in the first housing component. Here, the first housing component, e.g. implemented as a cartridge holder, and a cartridge assembled therein can be provided as a pre-fabricated housing assembly or as a dedicated cartridge-cartridge holder combination.

In either way, it can be assured or provided that a particular medicament provided in a particular cartridge is unequivocally associated with a particular type of a first housing component, i.e. with a particularly mechanically encoded first housing component. In effect and with some examples, a cartridge provided with a particular medicament can be only accommodated in a correspondingly shaped first housing component equipped with a respective mechanical coding.

With further examples a pre-fabricated housing assembly or a dedicated cartridge-cartridge holder combination is commercially distributed by a pharmaceutical manufacturer. Here, the cartridge may be undetachably or irremovably fixed inside the first housing component and the pharmaceutical manufacturer provides a respective matching between a cartridge filled with a particular medicament and a suitable first housing component, which is mechanically encoded in accordance to the type of medicament located inside the cartridge.

According to a further aspect the present disclosure also relates to a kit of injection devices. The kit of injection devices comprises at least a first injection device comprising a first housing provided with a coding and a counter coding both of a first type and further comprises a second injection device with a second housing provided and equipped with a coding and a counter coding both of a second type non-matching with the respective counter coding or coding of the first type.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition. Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, numerous examples of injection devices with dedicated or coded housing components will be described in greater detail by making reference to the drawings, in which:

FIG. 1 schematically illustrates an example of a drug delivery device,

FIG. 2 shows an example of an exploded view of the drug delivery device of FIG. 1,

FIG. 3 is a perspective illustration of first and second housing components before mutual assembly,

FIG. 4 is a perspective illustration of FIG. 3 with the first and second housing components mutually assembled,

FIG. 5 shows an isolated perspective illustration of the counter fastening element,

FIG. 6 is a perspective and cross-sectional view of the counter fastening element assembled to the sidewall of the second housing component,

FIG. 7 shows a perspective cross-section through the counter fastening element,

FIG. 8 a longitudinal cross-section through the second connecting end of the second housing component,

FIG. 9 is a perspective illustration of first and second connecting ends with the counter fastening element in the release position,

FIG. 10 is a perspective illustration of first and second connecting ends with the counter fastening element in the locking position,

FIG. 11 is a side view of the housing of the injection device,

FIG. 12 shows a cross-section along A-A according to FIG. 11 before assembly of first and second housing components,

FIG. 13 shows a cross-section A-A of FIG. 11 with first and second housing components mutually assembled and with the counter fastening element in the release position,

FIG. 14 shows a cross-section with the counter fastening element in the locking position,

FIG. 15 is a perspective illustration of the first and second connecting ends and the counter fastening element,

FIG. 16 is a further illustration of first and second connecting ends before mutual assembly without the counter fastening element,

FIG. 17 shows numerous cross-sectional profiles of different coding features,

FIG. 18 is an example of a first coding with a first coding feature,

FIG. 19 is an example of a second coding with a second coding feature,

FIG. 20 is an example of a third mechanical coding with a third coding feature

FIG. 21 is a further example of a mechanical coding with a first type of a mechanical coding feature,

FIG. 22 is a further example of a mechanical coding,

FIG. 23 is a further example of a mechanical coding,

FIG. 24 is a perspective illustration of a further example of a mechanical coding and a mechanical counter coding,

FIG. 25 is a further perspective illustration of another example of a mechanical coding and a mechanical counter coding,

FIG. 26 is a further example of a mechanical coding and counter coding,

FIG. 27 is a further example of a mechanical coding and a mechanical counter coding and

FIG. 28 shows another example of a mechanical coding and a mechanical counter coding.

### DETAILED DESCRIPTION

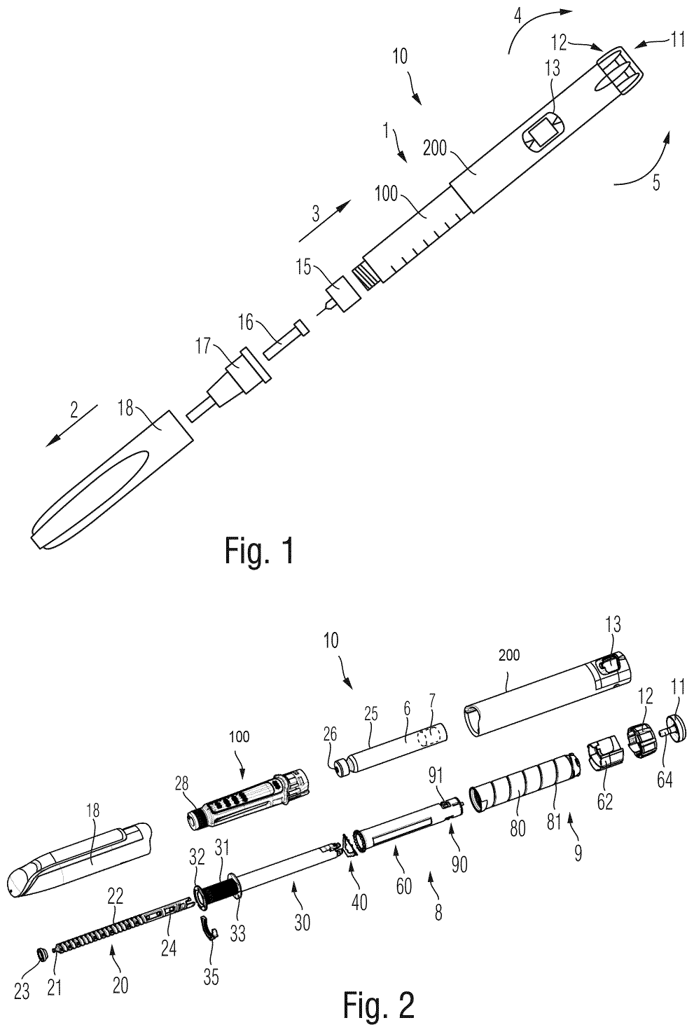

In FIGS. 1 and 2 only one of numerous examples of a handheld injection device is illustrated, that is generally usable in combination with a wearable electronic device. The device as shown in FIGS. 1 and 2 is a pre-filled disposable injection device that comprises a housing 10 to which an injection needle 15 can be affixed. The injection needle 15 is protected by an inner needle cap 16 and either an outer needle cap 17 or a protective cap 18 that is configured to enclose and to protect a distal section of the housing 10 of the injection device 1. The housing 10 comprises a first housing component 100 and a second housing component 200. The second housing component may form a main housing part configured to accommodate a drive mechanism 8 and/or a dose setting mechanism 9 as shown in FIG. 2. The first housing component 100 is configured as a cartridge holder. It may be permanently or releasably connected to the second housing component 200.

The first housing component 100 is typically configured to accommodate a cartridge 6 that is filled with a liquid medicament. The cartridge 6 comprises a cylindrically-shaped or tubular-shaped barrel 25 sealed in proximal direction 3 by means of a bung 7 located inside the barrel 25. The bung 7 is displaceable relative to the barrel 25 of the cartridge 6 in a distal direction 2 by means of a piston rod 20. A distal end of the cartridge 6 is sealed by a pierceable seal 26 configured as a septum and being pierceable by a proximally directed tipped end of the injection needle 15. The cartridge holder and hence the first housing component 100 comprises a threaded socket 28 at its distal end to threadedly engage with a correspondingly threaded portion of the injection needle 15. By attaching the injection needle 15 to the distal end of the first housing component 100 the seal 26 of the cartridge 6 is penetrated thereby establishing a fluid transferring access to the interior of the cartridge 6.

When the injection device 1 is configured to administer e.g. human insulin, the dosage set by a dose dial 12 at a proximal end of the injection device 1 may be displayed in so-called international units (IU, wherein 1 IU is the biological equivalent of about 45.5 μg of pure crystalline insulin (1/22 mg). The dose dial 12 may comprise or may form a dose dial.

As shown further in FIGS. 1 and 2, the housing 10, e.g. the second housing component 200 comprises a dosage window 13 that may be in the form of an aperture in the housing 10. The dosage window 13 permits a user to view a limited portion of a number sleeve 80 that is configured to move when the dose dial 12 is turned, to provide a visual indication of a currently set dose. The dose dial 12 is rotated on a helical path with respect to the housing 10 when turned during setting and/or dispensing or expelling of a dose.

The injection device 1 may be configured so that turning the dosage knob 12 causes a mechanical click sound to provide acoustical feedback to a user. The click sound is typically generated by a click noise generator 45. Generally, a click noise generator 45 may be implemented in various different ways. The number sleeve 80 mechanically interacts with a piston in the insulin cartridge 6. When the needle 15 is stuck into a skin portion of a patient, and when the trigger 11 or injection button is pushed, the dose displayed in display window 13 will be ejected from injection device 1. When the needle 15 of the injection device 1 remains for a certain time in the skin portion after the trigger 11 is pushed, the dose is actually injected into the patient's body. Ejection of a dose of the liquid medicament may also cause a mechanical click sound, which is however different from the click sound produced when using the dose dial 12. For this, the injection device one may comprise a separate, hence a second click noise generator (not illustrated).

In this embodiment, during delivery of the insulin dose, the dose dial 12 is turned to its initial position in an axial movement, that is to say without rotation, while the number sleeve 80 is rotated to return to its initial position, e.g. to display a dose of zero units.

The injection device 1 may be used for several injection processes until either the cartridge 6 is empty or the expiration date of the medicament in the injection device 1 (e.g. 28 days after the first use) is reached.

An example of the drive mechanism 8 is illustrated in more detail in FIG. 2. It comprises numerous mechanically interacting components. A flange like support of the housing 10 comprises a threaded axial through opening threadedly engaged with a first thread or distal thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod 20 rotates relative to the housing 10 thereby experiencing a distally directed advancing motion relative to the housing 10 and hence relative to the barrel 25 of the cartridge 6. As a consequence, the bung 7 of the cartridge 6 is displaced in distal direction 2 by a well-defined distance due to the threaded engagement of the piston rod 20 with the housing 10.

The piston rod 20 is further provided with a second thread 24 at its proximal end. The distal thread 22 and the proximal thread 24 are oppositely handed.

There is further provided a drive sleeve 30 having a hollow interior to receive the piston rod 20. The drive sleeve 30 comprises an inner thread threadedly engaged with the proximal thread 24 of the piston rod 20. Moreover, the drive sleeve 30 comprises an outer threaded section 31 at its distal end. The threaded section 31 is axially confined between a distal flange portion 32 and another flange portion 33 located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further comprises a radial recess or protrusion at its outer circumference to engage with a complementary-shaped recess or protrusion at an inside of the sidewall of the housing 10. In this way the last dose limiter 35 is splined to the housing 10, e.g. to the second housing component 200. A rotation of the drive sleeve 30 in a dose incrementing direction 4 or clockwise direction during consecutive dose setting procedures leads to an accumulative axial displacement of the last dose limiter 35 relative to the drive sleeve 30. There is further provided an annular spring 40 that is in axial abutment with a proximally facing surface of the flange portion 33. Moreover, there is provided a tubular-shaped clutch 60. At a first end the clutch 60 is provided with a series of circumferentially directed saw teeth. Towards a second opposite end of the clutch 60 there is located a radially inwardly directed flange.

Furthermore, there is provided a dose dial sleeve also denoted as number sleeve 80. The number sleeve 80 is provided outside of the spring 40 and the clutch 60 and is located radially inward of the housing 10. A helical groove 81 is provided about an outer surface of the number sleeve 80. The housing 10 is provided with the dosage window 13 through which a part of the outer surface of the number 80 can be seen. The housing 10 is further provided with a helical rib at an inside sidewall portion of an insert piece 62, which helical rib is to be seated in the helical groove 81 of the number sleeve 80. The tubular shaped insert piece 62 is inserted into the proximal end of the housing 10. It is rotationally and axially fixed to the housing 10. There are provided first and second stops on the housing 10 to limit a dose setting procedure during which the number sleeve 80 is rotated in a helical motion relative to the housing 10.

The dose dial 12 in form of a dose dial grip is disposed about an outer surface of the proximal end of the number sleeve 80. An outer diameter of the dose dial 12 typically corresponds to and matches with the outer diameter of the housing 10. The dose dial 12 is secured to the number 80 to prevent relative movement there between. The dose dial 12 is provided with a central opening.

The trigger 11, also denoted as dose button is substantially T-shaped. It is provided at a proximal end of the injection device 10. A stem 64 of the trigger 11 extends through the opening in the dose dial 12, through an inner diameter of extensions of the drive sleeve 30 and into a receiving recess at the proximal end of the piston rod 20. The stem 64 is retained for limited axial movement in the drive sleeve 30 and against rotation with respect thereto. A head of the trigger 11 is generally circular. The trigger side wall or skirt extends from a periphery of the head and is further adapted to be seated in a proximally accessible annular recess of the dose dial 12.

To dial a dose a user rotates the dose dial 12. With the spring 40, also acting as a click noise generator 45, and the clutch 60 engaged, the drive sleeve 30, the spring 40, the clutch 60 and the number sleeve 80 rotate with the dose dial 12. Audible and tactile feedback of the dose being dialed is provided by the spring 40 and by the clutch 60. Torque is transmitted through saw teeth between the spring 40 and the clutch 60. The helical groove 81 on the number sleeve 80 and a helical groove in the drive sleeve 30 have the same lead. This allows the number sleeve 80 to extend from the housing 10 and the drive sleeve 30 to climb the piston rod 20 at the same rate. At a limit of travel a radial stop on the number sleeve 80 engages either with a first stop or a second stop provided on the housing 10 to prevent further movement in a first sense of rotation, e.g. in a dose incrementing direction 4. Rotation of the piston rod 20 is prevented due to the opposing directions of the overall and driven threads on the piston rod 20.

The last dose limiter 35 keyed to the housing 10 is advanced along the threaded section 31 by the rotation of the drive sleeve 30. When a final dose dispensed position is reached, a radial stop formed on a surface of the last dose limiter 35 abuts a radial stop on the flange portion 33 of the drive sleeve 30, preventing both, the last dose limiter 35 and the drive sleeve 30 from rotating further.

Should a user inadvertently dial beyond the desired dosage, the injection device1, configured as a pen-injector allows the dosage to be dialed down without dispense of the medicament from the cartridge 6. For this the dose dial 12 is simply counter-rotated. This causes the system to act in reverse. A flexible arm of the spring or clicker 40 then acts as a ratchet preventing the spring 40 from rotating. The torque transmitted through the clutch 60 causes the saw teeth to ride over one another to create the clicks corresponding to dialed dose reduction. Typically, the saw teeth are so disposed that a circumferential extent of each saw tooth corresponds to a unit dose. Here, the clutch may serve as a ratchet mechanism.

As an alternative or in addition the ratchet mechanism 90 may comprise at least one ratchet feature 91, such as a flexible arm on the sidewall of the tubular-shaped clutch 60. The at least one ratchet feature 91 may comprise a radially outwardly extending protrusion e.g. on a free end of the flexible arm. The protrusion is configured to engage with a correspondingly shaped counter ratchet structure on an inside of the number sleeve 80. The inside of the number sleeve 80 may comprise longitudinally shaped grooves or protrusions featuring a saw-tooth profile.

During dialing or setting of a dose the ratchet mechanism 90 allows and supports a rotation of the number sleeve 80 relative to the clutch 60 along a second sense of rotation 5, which rotation is accompanied by a regular clicking of the flexible arm of the clutch 60. An angular momentum applied to the number sleeve 80 along the first sense of rotation for is unalterably transferred to the clutch 60. Here, the mutually corresponding ratchet features of the ratchet mechanism 90 provide a torque transmission from the number sleeve 80 to the clutch 60.

When the desired dose has been dialed the user may simply dispense the set dose by depressing the trigger 11. This displaces the clutch 60 axially with respect to the number sleeve 80 causing dog teeth thereof to disengage. However, the clutch 60 remains keyed in rotation to the drive sleeve 30. The number sleeve 80 and the dose dial 12 are now free to rotate in accordance with the helical groove 81.

The axial movement deforms the flexible arm of the spring 40 to ensure the saw teeth cannot be overhauled during dispense. This prevents the drive sleeve 30 from rotating with respect to the housing 10 though it is still free to move axially with respect thereto. The deformation is subsequently used to urge the spring 40 and the clutch 60 back along the drive sleeve 30 to restore the connection between the clutch 60 and the number sleeve 80 when the distally directed dispensing pressure is removed from the trigger 11.

The longitudinal axial movement of the drive sleeve 30 causes the piston rod 20 to rotate through the through opening of the support of the housing 10, thereby to advance the bung 7 in the cartridge 6. Once the dialed dose has been dispensed, the number sleeve 80 is prevented from further rotation by contact of at least one stop extending from the dose dial 12 with at least one corresponding stop of the housing 10. A zero dose position may be determined by the abutment of one of axially extending edges or stops of the number sleeve 80 with at least one or several corresponding stops of the housing 10.

The expelling mechanism or drive mechanism 8 as described above is only exemplary for one of a plurality of differently configured drive mechanisms that are generally implementable in a disposable pen-injector. The drive mechanism as described above is explained in more detail e.g. in WO2004/078239A1, WO 2004/078240A1 or WO 2004/078241A1 the entirety of which being incorporated herein by reference.

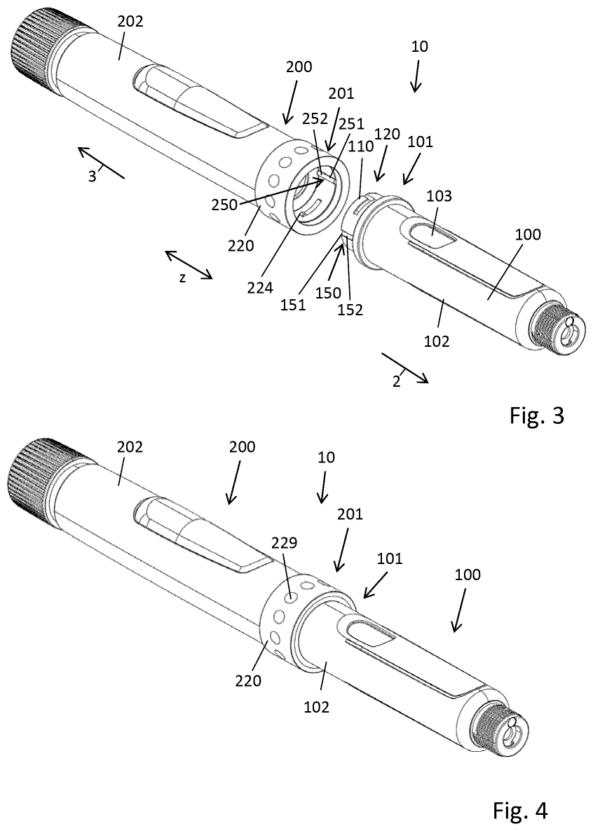

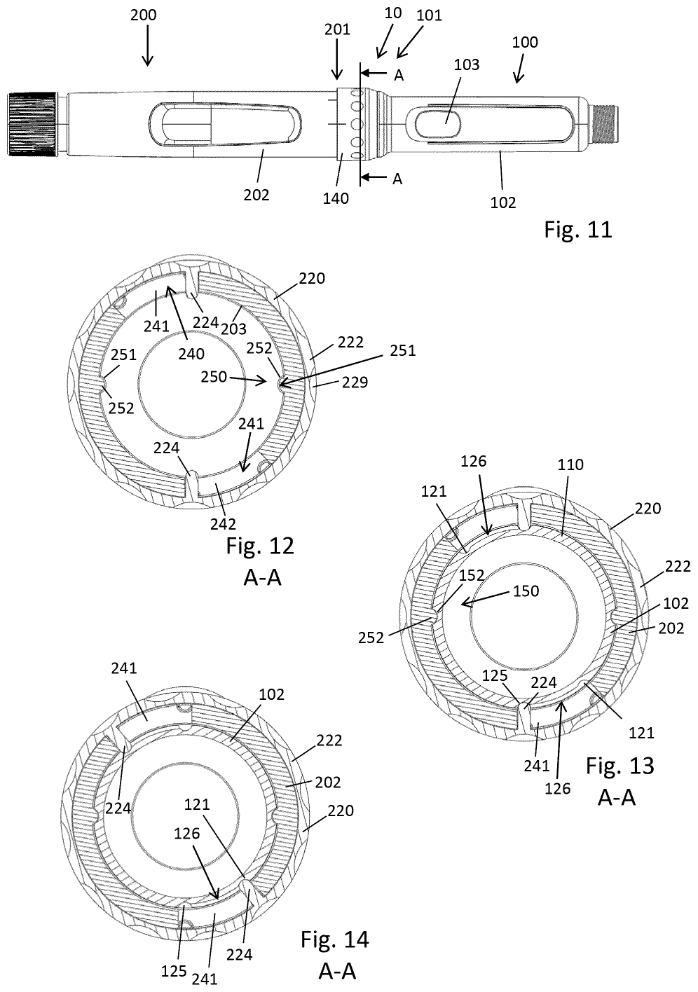

The housing 10 as illustrated in the numerous examples of FIGS. 3-16 comprises a first housing component 100 and a second housing component 200. The first housing component 100 is configured as a cartridge holder. It is sized and shaped to accommodate a cartridge 6 inside its hollow interior. The cartridge holder and hence the first housing component 100 comprises a first connecting end 101. The first connecting end 101 forms a proximal end of the first housing component 100. Correspondingly, the second housing component 200 comprises a second connecting end 201, typically at a distal end of the housing component 200.

The first connecting end 101 is mechanically connectable to the second connecting end 201. As illustrated, the first housing component 100 comprises an insert 110 forming the first connecting end 101. The second housing component 200 comprises a receptacle 210 shaped and sized to receive the insert 110. The receptacle 210 is radially confined by a sidewall 202 of the second housing component 200. The insert 110 is at least partially insertable into the receptacle 210 by a longitudinal sliding movement relative to the second housing component 200, in particular along the proximal direction 3.

The insert 110 forms a proximal end of the first housing component 100. The insert 110 comprises a proximal end face 112. Towards the distal direction 2 the insert 110 is confined by a flange section 115 protruding radially outwardly from the tubular shaped sidewall 102 of the first housing component 100 and hence also from a sidewall 102 of insert 110. The flange section 115 is structurally stiffened by a circumferential protruding radially outwardly extending rim 116 protruding from the outside surface 105 of the sidewall 102 of the first housing component 100.

In other words, the flange section 115 comprises the circumferential rim 116 extending all around the tubular shaped insert 110. Towards the proximal direction 3 the flange section 115 comprises an abutment or stop face 114 facing in proximal direction 3. The abutment or stop face 114 is configured to axially abut a distal end face 214 of a sidewall 202 of the second housing component 200. The sidewall 102 of the first housing component 100 may comprise a window 103, which can be implemented as a through recess intersecting the sidewall 102. The window 103 allows and supports visual inspection of the cartridge 6 and its content arranged inside the first housing component 100.

The receptacle 210 is open towards the distal direction 2. Insofar, the insert 110 of the first housing component 100 can be inserted into the receptacle 210 along the proximal direction 3 until a final assembly configuration has been reached. There is provided a fastening element 120 on the outside surface 105 of the insert 110 complementary shaped to a counter fastening element 220 rotationally or pivotally supported on the sidewall 202 that confines the receptacle 210.

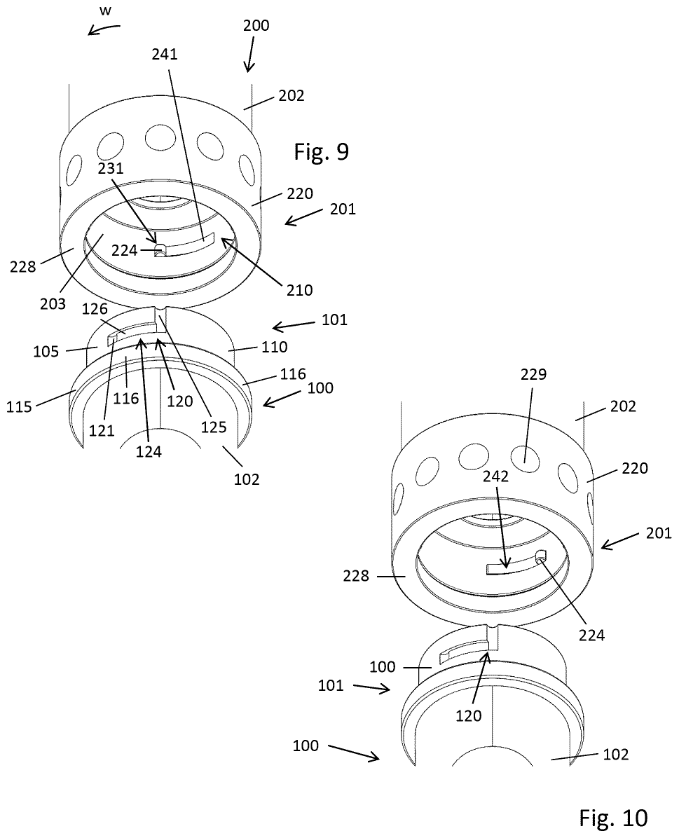

With the example of FIGS. 3-16 the fastening element 120 is provided on the insert 110. The fastening element 120 comprises a fastening groove 124. As illustrated in FIG. 9 the fastening groove 124 comprises a first groove portion 125 extending along the longitudinal direction (z). The second groove portion 126 extends along a circumferential direction (w). The second groove portion 126 merges into the first groove portion 125. The first groove portion 125 adjoins a longitudinal end face 112 of the first connecting end 101. The proximal end of the first groove portion 125 is located in the end face 112 of the insert 110. The second groove portion 126 is located at a predefined longitudinal distance from the longitudinal end face 112. The second groove portion 126 extends parallel to the longitudinal end face 112. In the illustrated example the second groove portion 126 extends substantially perpendicular to the first groove portion 125. Insofar the first and second groove portions 125, 126 form a L-shaped groove on the outside surface 105 of the insert 110.

The fastening groove 124 comprises a radial depth that is smaller than the thickness of the sidewall 102 of the insert 110. Insofar, the fastening groove 124 is void of a through opening extending through the sidewall 102. This improves stability and rigidity of the sidewall 102 and hence of the entire housing component 100.

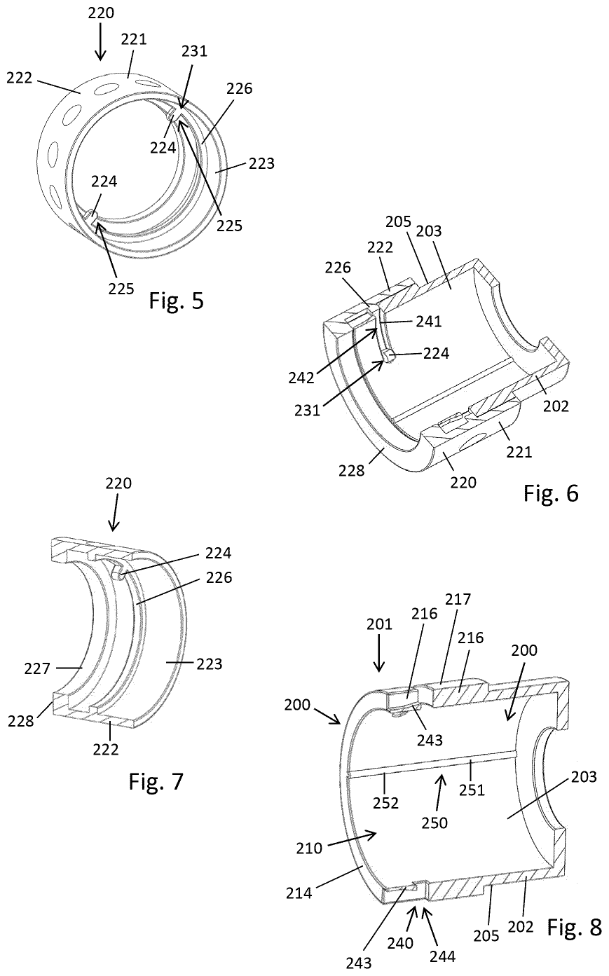

From an inside surface 203 of the receptacle 210 there protrudes a radially inwardly extending protrusion 224 of the counter fastening element 220. The protrusion 224 is sized and shaped to enter and to slide along the fastening groove 124. The counter fastening element 220 comprises a locking ring 221 or locking sleeve rotationally supported on the second connecting end 201. As illustrated, the counter fastening element 220 encloses and surrounds the sidewall 202 of the second connecting end 201 of the second housing component 200. It is axially or longitudinally fixed to the second housing component 200. The protrusion 224 protrudes radially inwardly from an inside surface 223 of the locking ring 221 as shown in FIG. 5.

Apparently, there are provided two or even more protrusions 224 extending radially inwardly on opposite sides of the inside surface 223 of the locking ring 221. As illustrated in FIG. 6 the protrusion 224 radially intersects a through recess 241 of a fastening structure 240 provided in or on the sidewall 202 of the second housing component 200. The through recess 241 comprises a circumferentially extending slot 242. The circumferential extent or circumferential width of the slot 242 typically matches with or corresponds to the circumferential extent of the second groove portion 126. The longitudinal extent of the slot 242 typically matches the longitudinal extent of the protrusion 224. In this way and when received in the slot 242 the protrusion 224 is hindered to move in longitudinal or axial direction relative to the second housing component 200. By way of the engagement of the slot 242 and the protrusion 224 the counter fastening element 220 can be axially constrained to the second housing component 200.

The counter fastening element 220 is rotationally supported on the sidewall 202 that confines the receptacle 210. As illustrated in FIG. 9 the counter fastening element 220 is in a release position. In this position or configuration the counter fastening element 220 allows and supports a longitudinal sliding movement of the first housing component 100 relative to the second housing component 200. In FIG. 10 the counter fastening element 220 has been rotated relative to the position as illustrated in FIG. 9. In FIG. 10 the counter fastening element 220 is in a locking position. The release position and the locking position as illustrated in FIGS. 9 and 10 are defined by the oppositely located circumferential end of the slot 242.

In order to provide a precise and slip free handling or movement and to provide a rather slip free rotation of the counter fastening element 220 the counter fastening element 220 comprises a gripping structure 229, e.g. in form of multiple depressions or recesses on the outside surface of its annular shaped sidewall 222.

Assembly and fixing of the first housing component 100 to the second housing component 200 is possible when the counter fastening element 220 is in the release position as illustrated in FIG. 9. Then, the protrusion 224 is aligned with the first groove portion 125. Only in this configuration, the insert 110 is insertable into longitudinal direction into the receptacle 210. During this insert motion the protrusion 224 slides along the first groove portion 125. Since there are provided two oppositely located protrusions 224 also the insert 110 comprises two oppositely located fastening elements 124.

The longitudinal insert motion is delimited and stopped when the flange section 115 of the first housing component 100 longitudinally abuts the distal end face 214 of the second housing component 200 or abuts a distal end face 228 of the counter fastening element 220. Then, the proximally facing stop face 114 of the flange section 115 and hence of the rim 116 gets in longitudinal abutment with the second housing component 200 or with the counter fastening element 220. When reaching such a final insert position the protrusion 224 will be aligned, e.g. circumferentially aligned with the second groove portion 126. Hence, only and when arriving in the final insert position the protrusion 224 is allowed to move or to slide along the second groove portion 126. This is achieved by rotating or pivoting the counter fastening element 220 relative to the sidewall 202, which motion which becomes apparent by a comparison of FIGS. 9 and 10.

The protrusion 224 comprises or constitutes a counter snap element 231 configured to engage with a complementary-shaped snap element 121 of the fastening element 120. The counter snap element 231 is provided by the free end of the protrusion 224. The snap element 121 is provided by a depression or recess at the circumferential or longitudinal end of the second groove portion 126 facing away the first groove portion 125. So when arriving in the locking position the counter snap element 231 snaps into the snap element 121. In this way, the counter fastening element 220 is rotationally secured to the fastening element 120.

The perpendicular orientation of the first groove portion 125 and the second groove portion 126 provides the further benefit that a rotation of the counter fastening element 220 from the release position as illustrated in FIG. 9 into the locking position as illustrated in FIG. 10 is only and exclusively supported and possible when the insert 110 has reached the final insert position inside the receptacle 210. In situations where the insert 110 has not yet reached the final insert position, in which the flange section 115 longitudinally abuts the distal end face 214 of the housing component 204 or the distal end face 228 of the counter fastening element 220 rotation of the counter fastening element 220 towards and into the locking position will be blocked because the protrusion 224 is not aligned with the second groove portion 126 and is still located in the first groove portion 125.

The L-shaped groove 124 and the further feature that the width of the second groove portion as seen in longitudinal direction matches the longitudinal extent of the protrusion 224 provides a safety feature that a mutual fastening and fixing of the fastening element 120 and the counter fastening element 220 is only and exclusively possible when the insert 110 has reached the final insert position.

Moreover, the counter snap element 231 mechanically engaging with the snap element 121 provides audible and haptic feedback to the user that the counter fastening element 220 has reached the locking position. This provides a further safety feature for establishing a mutual fixing of the first and second housing components 100, 200.

The mechanical engagement of the snap element 121 and the counter snap element 231 is typically accompanied by an elastic deformation of at least one of the snap element 121 and the counter snap element 231 in radial direction. Typically, both fastening elements 120 are each provided with a snap element 121. Correspondingly, also the two oppositely located protrusions 224 are provided with or form respective counter snap elements 231. In order to enable and to achieve a rather smooth snap fit configuration the snap element 131 may comprise a somewhat concave shaped recess. Complementary, the counter snap element 231 and hence the protrusion 224 comprises a somewhat convex-shaped free end as seen in the transverse or circumferential profile of the protrusion 224.

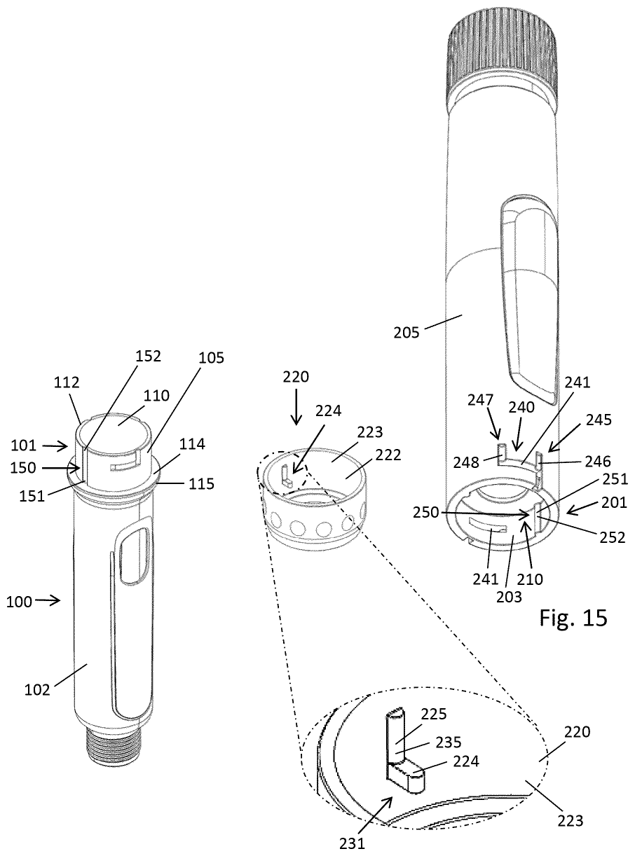

As becomes further apparent from FIG. 15 the insert 110 is provided with a mechanical coding 150 and the receptacle 210 is provided with a complementary-shaped mechanical counter coding 254. The mechanical coding 150 comprises a mechanical coding feature 151. In the illustrated example the coding feature 151 comprises a longitudinally extending coding slot 152 extending along the longitudinal extent of the insert 110. A proximal end of the coding slot 152 adjoins the proximal end face 112 of the insert 110.

Complementary to the coding feature 151 there is provided a counter coding feature 251 on the inside 203 of the receptacle 210. The counter coding feature 251 comprises a counter coding projection 252. The counter coding projection 252 matches in size and shape with the coding slot 152. Insofar the coding slot 152 and the counter coding projection 252 form or establish a rotational interlock between the insert 110 and the receptacle 210 when the insert 110 is longitudinally inserted into the receptacle 210.

Insertion of the insert 110 into the receptacle 210 is only possible and supported when the mechanical coding 150 matches the mechanical counter coding 250. This requires that the number of coding features 150 matches the number of counter coding features 251. Moreover, the geometry and shape of the coding features 151 has to match the geometry and shape of the counter coding features 251. Additionally, the circumferential position as well as the circumferential and radial extent of the coding features 151 has to match the respective position and extent of the counter coding feature 251.

As illustrated in FIG. 15 the counter coding feature 251, in particular the counter coding projection 252 comprises an elongated radially inwardly protruding rib that matches in size and shape with the coding slot 152. In this way and upon inserting the insert 110 into the receptacle 210 a keyed engagement between the mechanical coding 150 and the complementary shaped mechanical counter coding 250 is established. Thus, the insert 110 is hindered to rotate relative to the receptacle 210. By way of the rotational interlock as provided by the mutually corresponding mechanical coding 150 and mechanical counter coding 250 the insert motion of the insert 110 into the receptacle 210 is purely axial or longitudinal. The insert motion is void of a rotation.

As becomes apparent from FIG. 15 the mechanical coding 150 comprises two coding features 151 located on opposite sides of the outside surface 105 of the insert 110. Complementary, also the counter coding 250 comprises two oppositely located counter coding features 251. The mutual engagement of the mechanical coding 250 with the mechanical counter coding 150 is also apparent from the cross-section of FIGS. 13 and 14.

In FIG. 12, the insert 110 has not yet been inserted into the receptacle 210. The counter fastening element 220 is in the release position. As illustrated, the protrusion 224 protrudes radially inwardly from the inside surface 203 of the sidewall 202. It extends through the through recess 241 and hence through the slot 242 of the fastening structure 240. In FIG. 13 the insert 110 has been inserted into the receptacle 210. There, the insert 110 is in the final insert position. Here, the second groove portion 126 tangentially or circumferentially aligns with the protrusion 224. In the release position of the counter fastening element 220 as illustrated in FIG. 13 the free end of the protrusion 224 is located in the first groove portion 125. By rotating the counter fastening element 220 counterclockwise the protrusion 224 slides along the second groove portion and engages the snap element 121 as illustrated in FIG. 14.

As illustrated in greater detail in FIGS. 5-10 the locking ring 221 comprises an annular sidewall 222 with an inside surface 223 from which the protrusions 224 extend radially inwardly. The protrusion 224 itself may provide a counter retainer 225 by way of which the counter fastening element 220 can be at least rotationally fixed to the second housing component 200 or to the first housing component, namely when the protrusion 224 forms a snap fit with the snap element 121.

There is further provided a radially inwardly extending rib 226 of annular shape extending all around the inside circumference of the sidewall 222. The rib 226 as illustrated in FIG. 6 may contribute to the rotational support of the counter fastening element 220 on the outside surface 205 of the sidewall 202. The rib 226 may longitudinally engage with side edges of the through recess 241 or of the circumferential slot 242. The annular rim 216 provides a rotational support for the counter fastening element 220 on the sidewall 202 of the second housing component 200. In addition, the outside surface 205 of the sidewall 202 may be also provided with an annular groove matching the shape and size with the annular rib 226.

The locking ring 221 further comprises a radially inwardly extending flange section 227. The flange section 227 forms a kind of a stiffening rim at the distal end of the locking ring 221. The flange section 227 is of annular shape and protrudes radially inwardly from the sidewall 222 of the locking ring 221. The flange section 227 comprises a proximally facing surface that longitudinally abuts the distal end face 214 of the sidewall 202 of the second housing component. In this way there can be provided a supplemental rotational support for the counter fastening element 220. The flange section 227 comprises a distal end face 228, which forms or constitutes a distal end face of the second housing component 200 when the counter fastening element 220 is assembled and fixed to the sidewall 202. When mutually assembled, the flange section 227 is located distally from the longitudinal end face 214 of the sidewall 202.

As it is apparent in greater detail from a combination of FIGS. 8, 15 and 16 the sidewall 202 of the second housing component 200 comprises a fastening structure 240 with an assembly groove 244. The assembly groove 244 adjoins the distal end face 214 of the sidewall 202 and merges into the through recess 241, which is located at a proximal offset from the distal end face 214. As illustrated in FIG. 8, the through recess 241 may be provided in a radially widened rim 216 provided at the second connecting end 201 of the second housing component 200. The rim 216 may be integrally formed with the sidewall 202. It may provide a radially thickened sidewall portion. The annular rim 216 comprises a radially outwardly extending stepped portion protruding radially outwardly from a proximal part of the sidewall 202 of the second housing component 200. On the outside surface of the rim 216 there is provided an even shaped support surface 217 on which the counter fastening element 220 may smoothly slide in circumferential direction (w).

As becomes apparent from FIG. 8 the assembly groove 244 comprises a radial depth that is smaller than the thickness of the sidewall 202 in this particular region. Insofar, the assembly groove 244 is void of a through opening extending through the sidewall 202. Only where the assembly groove 244 merges into the circumferentially extending slot 242 there is provided a through recess 241 in the sidewall 202. In this way the distal end face 214 of the sidewall 202 is void of a slot or recess extending entirely through the sidewall 202. Accordingly, the stability and rigidity of the second connecting end 201 can be improved. The assembly groove 244 is provided with a snap element 243 located in the assembly groove 244, e.g. on the bottom of the assembly groove 244.

The snap element 243 comprises a beveled gliding or ramp surface extending radially outwardly towards the slot 242. The snap element 243 provides a ramp for the protrusion 224 of the counter fastening element 220. For mounting the counter fastening element 220 onto the sidewall 202 it is required to align the protrusions 224 with the assembly grooves 244 of the oppositely located fastening structures 240. Then, and through a proximally directed sliding movement of the counter fastening element 220 relative to the second housing component 200 the protrusions 224 slide along the ramp and hence along the snap elements 243. This motion is accompanied by an elastic deformation of at least one of the protrusions 224 and the ramp or snap elements 243. Once the protrusions 224 have longitudinally passed the snap elements 243 they may 224 audibly and/or haptically engage with the through recess 241. The snap elements 243 and hence the ramp provided in the assembly groove 244 prevent a disassembly of the counter fastening element 220 from the fastening structure 240.

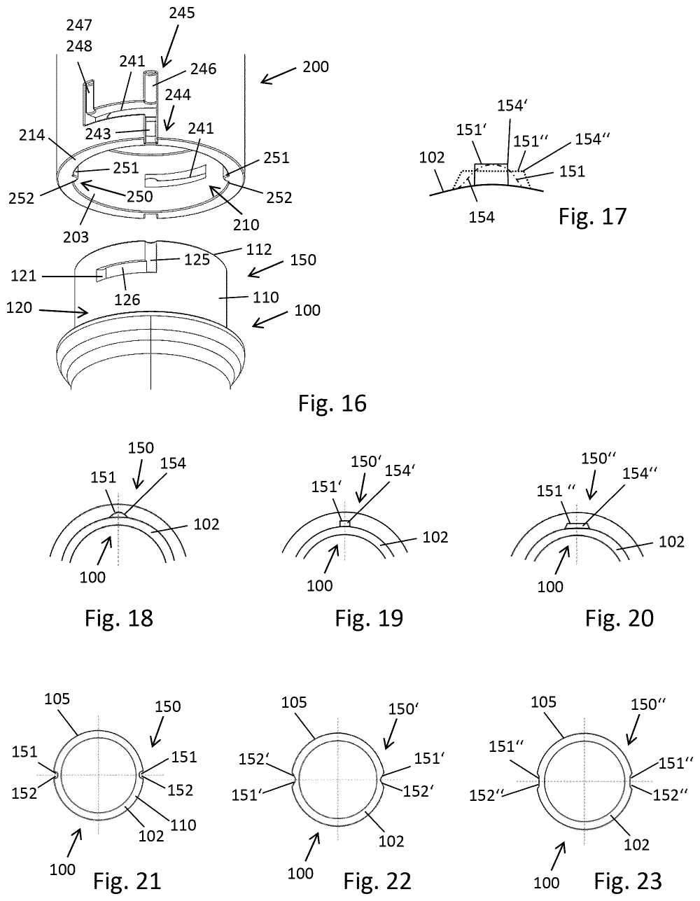

As further illustrated in FIGS. 15 and 16 the fastening structure 240 comprises a first retainer 245 and a second retainer 247. On the outside surface 205 of the sidewall 202 the retainers 245, 247 merge and adjoin the through recess 241. The first and second retainers 245 and 247 are located at a circumferential distance from each other. In the presently illustrated example, the first retainer 245 is longitudinally aligned with the assembly groove 244. It extends proximally from the through recess 241 while the assembly groove 244 extends distally from the through recess. As illustrated in greater detail in FIG. 15 there is not only provided a protrusion 224 on the inside surface 223 of the sidewall 222 of the counter fastening element 220 but there is further provided a counter retainer 225. The counter retainer 225 comprises a radially inwardly extending protrusion 235 that matches in size and shape with a complementary shaped recesses 246, 248 of the first and second retainers 245, 247.

In this way and when the counter fastening element 220 is in the release position the counter retainer 225 is engaged with the first retainer 245. Hence, the protrusion 235 is engaged with the recess 246. Through the mutual engagement of the first retainer 245 with the counter retainer 235 the counter fastening element 220 can be locked or fixed in the release position. Here, the retainer 245 and the counter retainer 225 form and to establish a snap fit connection between the counter fastening element 220 and the second housing components 200.

For transferring or rotating the counter fastening element 220 from the release position into the locking position a well-defined torque or force must be applied to the counter fastening element 220 in order to overcome and abrogate the snap fit engagement between the first retainer 245 and the counter retainer 225. Once the counter fastening element 220 is subject to a respective force effect above a break loose force as provided by the snap fit engagement, the counter fastening element 220 is allowed to rotate towards the locking position. When arriving in the locking position the counter retainer 225 may engage the second retainer 247 and hence the recess 248.

In the locking position the counter fastening element 220 is rotationally locked to the fastening structure 240 and hence to the sidewall 202 by the snap fit engagement of the counter retainer 225 with the second retainer 247. Simultaneously or concurrently the protrusion 224, hence the counter snap element 231 is in snap fit engagement with the snap element 121. In this way, a twofold snap fit engagement of the counter fastening element 222 to the sidewall 202 and to the insert 110 can be provided. By way of such a twofold snap fit engagement a break loose force required for releasing the counter fastening element 220 from the locking position may be larger than the break loose force required for releasing the counter fastening element 220 from the release position.

With the example of FIG. 16 the mechanical coding 150 comprises two oppositely located coding features 151 (not illustrated) matching in shape and geometry with complementary shaped counter coding features 251 of the counter coding 250 as provided on the inside surface 203 of the receptacle 210. Here, the coding features 151 each comprise a longitudinally extending coding slot 152 as illustrated for instance in FIG. 16. Complementary, the mechanical counter coding 250 comprises two oppositely located counter coding projections 252.

In FIGS. 17-20 there are illustrated numerous examples of different coding features 151, 151′, 151″. The coding feature 151 of FIG. 18 comprises a radially outwardly extending coding projection 154. The coding projection 154 and hence the coding feature 151 forms a mechanical coding 150 of a first type. The coding projections 154 is of knob-like, convex or rounded shape. The further example as illustrated in FIG. 19 shows a mechanical coding 150′ of a second type. The mechanical coding 150′ comprises a coding feature 151′ of a second type. The coding feature 151′ also comprises a coding projection 154′. The coding projections 154′ of the second type differ in shape and geometry as well as in circumferential and/or radial size from the coding projections 154 of the first type.

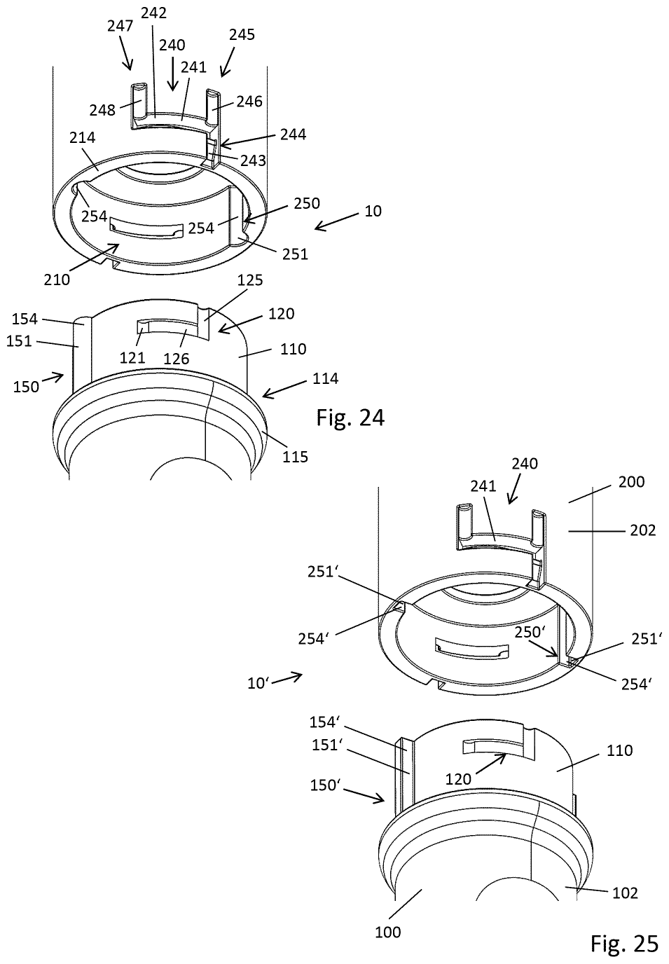

The coding feature 151 of the first type is also illustrated in FIG. 24. The counter coding feature 251 of the first type comprises a counter coding slot 254 in the sidewall 202 that matches the profile, position and size of the coding feature 151 of the first type.

The coding feature 151′ of the second type is complementary shaped to a respective counter coding feature 251′ of the second type as illustrated in FIG. 25. The position of the coding features 151, 151′ may be somewhat identical. But here, the coding feature 151 of the first type distinguishes from the coding feature 151′ of the second type by its shape. Hence, the coding projections 154′ of the mechanical coding 150′ of the second type is of rectangular or trapezoidal shape. As illustrated in the overlay of FIG. 17 the radial extent and/or the circumferential width of the coding feature 151′ is larger than the radial and/or circumferential extent of the coding feature 151.

Insofar the coding feature 151 of the first type is unable to pair with a counter coding feature 251′ of the second type; and vice versa.

The combination of FIGS. 24 and 25 shows a kit of two housing 10, 10′, wherein each housing 10 comprises a first housing component 100 and a second housing component 200. Here, the coding 150 of the first housing 10 as illustrated in FIG. 24 does not match the counter coding 250′ of the second housing 10′ as illustrated in FIG. 25; and vice versa.

The same may apply to the coding feature 151″ of a third type. As illustrated in FIGS. 17 and 20 the coding feature 151″ comprises a coding projection 154″ of a third type. The coding projections 154″ differ in size and shape from each of the other two coding projections 154, 154′. The coding projections 154″ and hence the coding features 151″ comprise a circumferential extent that is larger than the circumferential extent of any of the other coding features 151, 151′.

Similarly and as illustrated in FIGS. 21-23 the mechanical coding 150 may also comprise numerous coding features 151 implemented as coding slots 152 on the outside surface 105 of the sidewall 102 of the first housing component 100. In FIG. 21 a mechanical coding 150 of a first type is illustrated. The mechanical coding 150 comprises two coding features 151 distributed along the outer circumference of the insert 110. The coding features 151 are symmetric. They are provided at diametrically oppositely located side wall sections. The coding features 151 each comprise a coding slot 152. The coding slot 152 is of a concave or of a rounded shape.

The coding features 151 as provided at different circumferential positions on the outside surface 105 of the insert 110 and are equal in shape. With the further example of a mechanical coding 150′ of a second type according to FIG. 22 the position and number of coding features 151′ is identical to the position and number of coding features 151 of the first type. But there, the coding features 151′ each comprise a V-shaped notch 152′ forming a respective coding slot 152′. With the further example of FIG. 23 a mechanical coding 150″ of a third type is illustrated. There, the number and position of respective coding features 151″ is equal to the number and position of the coding features 151 of the mechanical coding 150 of the first type and with the number and position of the coding features 152′ of the mechanical coding 150′ of the second type.

Again, the shape and cross-section of the coding features 151″ differs from the respective shape and geometry of the coding features 151, 151′. Respective counter coding features 251, 251′, 251″ are not particularly illustrated here. The respective counter coding features 251, 251′, 251″ are complementary shaped to the coding features 151, 151′ and 151″, respectively.

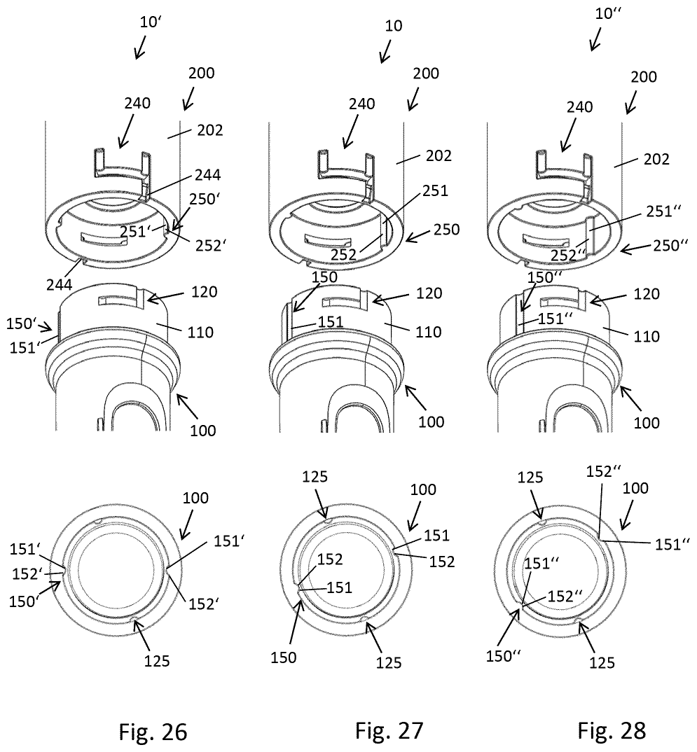

With the further examples as illustrated in FIGS. 26-28 a variation of a coding is achieved by modifying the circumferential position of the coding features relative to the fastening element 120. The further examples of FIGS. 26-28 are illustrative of three different types of housings 10, 10′ and 10″, wherein the coding 150 of a first housing 10 distinguishes from the codings 150′, 150″ of the second and third housings 10′, 10″, respectively. Insofar, the housings 10, 10′, 10″ represent a kit of housings comprising at least three individual housings 10, 10′, 10″. The first housing 10 is provided with a first mechanical coding 150 and with a first mechanical counter coding 250. The second housing 10′ is provided with a second mechanical coding 150′ and with a second mechanical counter coding 250′. The third housing 10″ is provided with a third mechanical coding 150″ and with a third mechanical counter coding 250″.

The first mechanical coding 150 does not match with any of the second or third mechanical counter codings 250′, 250″. Likewise, the second mechanical coding 150′ does not match with any of the first or third mechanical counter codings 250, 250″; and vice versa.

In the illustrated example, the mechanical coding 150 of the first type as illustrated in FIG. 27 comprises two coding features 151 diametrically opposite to each other. The coding features 151 are arranged at a 90° circumferential offset angle with respect to the fastening element 120, in particular with respect to the first groove portion 125 of the insert 110. With another mechanical coding 150′ of a second type as illustrated in FIG. 26 the circumferential position of the coding features 151′ relative to the fastening element 120 and relative to the first groove portion 125 of the fastening element 120 has changed.

Here and compared to the mechanical coding 150 of FIG. 27 the position of the coding features 151′ relative to the fastening element 120 has been shifted clockwise compared to the respective relative position of the coding features 151.

In FIG. 28 another configuration of a coding variation is illustrated. There, the mechanical coding 150″ of a second type distinguishes from the mechanical coding 150 of the first type by arranging the coding features 151″ at a counterclockwise offset from the position of the coding features 151 of the mechanical coding 150 of the first type relative to the fastening element 120.

With other non-illustrated examples the relative circumferential position of the first coding feature 151 of a first mechanical coding 150 relative to a second coding feature 151 of the same mechanical coding 150 may be varied. Moreover, and according to further examples, different codings 150 or counter codings 250 can be provided by implementing numerous coding features 151 and counter coding features 251 for each of the codings 150 or counter codings 250, wherein coding features 151 belonging to the same mechanical coding 150 are of different size or shape.

Separating the mechanical coding 150 from the fastening element 120 and the counter fastening element 220 may be of particular benefit to use one and the same fastening element 120 and counter fastening element 220 to provide a mutual fastening and fixing of the first and second housing components 100, 200 of various housings of different type. For providing different codings and counter codings only the position, shape, geometry or extent of the coding features 151 and the complementary-shaped counter coding features 251 will have to be modified. This way, a modification of the coding features 151 and counter coding features 251 has no particular influence on the fastening and fixing mechanism for fastening and fixing the first and second housing components 100, 200 to each other.

With the presently illustrated examples the insert 110 is provided on the first housing component 100 and the receptacle 210 is provided in the second housing component 200. There are numerous further examples conceivable and within the disclosure of the present application, wherein the insert is provided on the second housing component and wherein the correspondingly-shaped receptacle is provided on the first housing component. Likewise, the specific implementation of radially protruding and radially recessed features, as described in connection with the projection and the groove or in connection with the fastening element and counter fastening element may be interchanged and may be thus provided and implemented in an inverted way compared to the presently shown examples.

### Reference Numbers

1 injection device
2 distal direction
3 proximal direction
4 dose incrementing direction
5 dose decrementing direction
6 cartridge
7 bung
8 drive mechanism
9 dose setting mechanism
10 housing
11 trigger
12 dose dial
13 dosage window
14 cartridge holder
15 injection needle
16 inner needle cap
17 outer needle cap
18 protective cap
20 piston rod
21 bearing
22 first thread
23 pressure foot
24 second thread
25 barrel
26 seal
28 threaded socket
30 drive sleeve
31 threaded section
32 flange
33 flange
35 last dose limiter
40 spring
60 clutch
62 insert piece
64 stem
80 number sleeve
81 groove
90 ratchet mechanism
91 ratchet feature
100 housing component
101 connecting end
102 sidewall
103 window
105 outside surface
110 insert
112 end face
11 stop face
115 flange section
116 rim
120 fastening element
121 snap element
124 fastening groove
125 groove portion
126 groove portion
150 mechanical coding
151 coding feature
152 coding slot
154 coding projection
200 housing component
201 connecting end
202 sidewall
203 inside surface
205 outside surface
210 receptacle
214 end face
216 rim
217 support surface
220 counter fastening element
221 locking ring
222 sidewall
223 inside surface
224 protrusion
225 counter retainer
226 rib
227 flange section
228 end face
229 gripping structure
231 counter snap element
235 protrusion
240 fastening structure
241 through recess
242 slot
243 snap element
244 assembly groove
245 retainer
246 recess
247 retainer
248 recess
250 mechanical counter coding
251 counter coding feature
252 counter coding projection
254 counter coding slot

1. A housing of a drug delivery device, the housing comprising:
a first housing component configured to accommodate a cartridge filled with a medicament and comprising a first connecting end;
a second housing component configured to accommodate a drive mechanism of the drug delivery device and comprising a second connecting end;
an insert provided on one of the first connecting end and the second connecting end;
a receptacle confined by a sidewall and provided on the other one of the first connecting end and the second connecting end, wherein the insert is insertable into the receptacle along a longitudinal direction for mutually fastening the first housing component and the second housing component;
a fastening element provided on the insert;
a counter fastening element complementary shaped to the fastening element and rotationally supported on the sidewall between a locking position and a release position;
a mechanical coding provided on the insert and comprising a coding feature; and
a mechanical counter coding provided in the receptacle and comprising a counter coding feature,
wherein when the mechanical coding does not match the mechanical counter coding, the mechanical coding and the mechanical counter coding are operable to prevent at least one of:
an engagement of the fastening element with the counter fastening element, and
an insertion of the insert into the receptacle.

2. The housing according to claim 1, wherein one of the fastening element and the counter fastening element comprises a radial protrusion, and wherein the other one of the fastening element and the counter fastening element comprises a fastening groove.

3. The housing according to claim 2, wherein the fastening groove comprises a first groove portion and a second groove portion, wherein the first groove portion extends along the longitudinal direction, and wherein the second groove portion extends along a circumferential direction and merges into the first groove portion.

4. The housing according to claim 3, wherein the first groove portion adjoins a longitudinal end face of one of the first connecting end and the second connecting end.

5. The housing according to claim 3, wherein the second groove portion merges into the first groove portion at a longitudinal distance from the longitudinal end face of one of the first connecting end and the second connecting end.

6. The housing according to claim 1, wherein the fastening element comprises a snap element, and wherein the counter fastening element comprises a counter snap element to engage with the snap element when the counter fastening element reaches the locking position.

7. The housing according to claim 1, wherein the sidewall comprises a first retainer operable to fix the counter fastening element in the release position.

8. The housing according to claim 7, wherein the sidewall comprises a second retainer operable to fix the counter fastening element in the locking position.

9. The housing according to claim 7, wherein the counter fastening element comprises a counter retainer complementarily shaped to at least one of the first retainer and the second retainer and configured to form a snap fit connection with at least one of the first retainer and the second retainer.

10. The housing according to claim 9, wherein one of the first retainer, the second retainer, and the counter retainer comprises a radial protrusion, and wherein the other one of the first retainer, the second retainer, and the counter retainer comprises a radial recess complementarily shaped to the radial protrusion.

11. The housing according to claim 1, wherein the fastening element comprises the fastening groove provided on an outside surface of the insert.

12. The housing according to claim 1, wherein one of the coding feature and the counter coding feature comprises a radial coding projection, and wherein the other one of the coding feature and the counter coding feature comprises a longitudinally extending coding slot shaped to slidably receive the radial coding projection.

13. The housing according to claim 1, wherein the mechanical coding is defined by at least one of:
a number of coding features;
a circumferential position of coding features on the insert;
a circumferential extent of coding features on the insert;
a radial extent of coding features on the insert; and
a cross-sectional geometry or shape of coding features in a plane transverse to the longitudinal direction.

14. An injection device for injecting a dose of a medicament, the injection device comprising:
a housing comprising:
a first housing component configured to accommodate a cartridge filled with a medicament and comprising a first connecting end;
a second housing component configured to accommodate a drive mechanism of the drug delivery device and comprising a second connecting end;
an insert provided on one of the first connecting end and the second connecting end;
a receptacle confined by a sidewall and provided on the other one of the first connecting end and the second connecting end, wherein the insert is insertable into the receptacle along a longitudinal direction for mutually fastening the first housing component and the second housing component;
a fastening element provided on the insert;
a counter fastening element complementarily shaped to the fastening element and rotationally supported on the sidewall between a locking position and a release position;
a mechanical coding provided on the insert and comprising a coding feature; and
a mechanical counter coding provided in the receptacle and comprising a counter coding feature,
wherein when the mechanical coding does not match the mechanical counter coding, the mechanical coding and the mechanical counter coding are operable to prevent at least one of:
an engagement of the fastening element with the counter fastening element, and
an insertion of the insert into the receptacle; and
the cartridge, wherein the cartridge is arranged inside the housing and comprises a barrel filled with a medicament and sealed in a proximal longitudinal direction by a movable bung; and
the drive mechanism, wherein the drive mechanism is arranged inside the housing and comprises a piston rod operable to exert a distally directed dispensing force onto the bung of the cartridge.

15. A kit comprising:
a first housing of a drug delivery device, the first housing comprising:
a first housing component configured to accommodate a first cartridge filled with a first medicament and comprising a first connecting end;
a second housing component configured to accommodate a first drive mechanism of the drug delivery device and comprising a second connecting end;
a first insert provided on one of the first connecting end and the second connecting end;
a first receptacle confined by a first sidewall and provided on the other one of the first connecting end and the second connecting end, wherein the first insert is insertable into the first receptacle along a first longitudinal direction for mutually fastening the first housing component and the second housing component;
a first fastening element provided on the first insert;
a first counter fastening element complementarily shaped to the first fastening element and rotationally supported on the first sidewall between a first locking position and a first release position;
a first mechanical coding provided on the first insert and comprising a first coding feature; and
a first mechanical counter coding provided in the first receptacle and comprising a first counter coding feature,
wherein when the first mechanical coding does not match the first mechanical counter coding, the first mechanical coding and the first mechanical counter coding are operable to prevent at least one of:
a first engagement of the first fastening element with the first counter fastening element, and
a first insertion of the first insert into the first receptacle;
a second housing of the drug delivery device, the second housing comprising:
a third housing component configured to accommodate a second cartridge filled with a second medicament and comprising a third connecting end;
a fourth housing component configured to accommodate a second drive mechanism of the drug delivery device and comprising a fourth connecting end;
a second insert provided on one of the third connecting end and the fourth connecting end;
a second receptacle confined by a second sidewall and provided on the other one of the third connecting end and the fourth connecting end, wherein the second insert is insertable into the second receptacle along a second longitudinal direction for mutually fastening the third housing component and the fourth housing component;
a second fastening element provided on the second insert;
a second counter fastening element complementarily shaped to the second fastening element and rotationally supported on the second sidewall between a second locking position and a second release position;
a second mechanical coding provided on the second insert and comprising a second coding feature; and
a second mechanical counter coding provided in the second receptacle and comprising a second counter coding feature,
wherein when the second mechanical coding does not match the second mechanical counter coding, the second mechanical coding and the second mechanical counter coding are operable to prevent at least one of:
a second engagement of the second fastening element with the second counter fastening element, and
a second insertion of the second insert into the second receptacle;
wherein the first coding feature of the first housing distinguishes from the second coding feature of the second housing with regard to at least one of:
a number of coding features,
a circumferential position on the first and second inserts,
a circumferential extent on the first and second inserts,
a radial extent on the insert first and second inserts, and
a cross-sectional geometry or shape in a plane transverse to the longitudinal direction.

16. The housing according to claim 1, further comprising the cartridge.

17. The injection device according to claim 14, wherein one of the fastening element and the counter fastening element comprises a radial protrusion, and wherein the other one of the fastening element and the counter fastening element comprises a fastening groove.

18. The injection device according to claim 14, wherein the fastening element comprises a snap element, and wherein the counter fastening element comprises a counter snap element to engage with the snap element when the counter fastening element reaches the locking position.

19. The injection device according to claim 14, wherein the sidewall comprises a first retainer operable to fix the counter fastening element in the release position.

20. The injection device according to claim 14, wherein the fastening element comprises the fastening groove provided on an outside surface of the insert.
